(19) United States
(12) Patent Application Publication
(10) Pub. No.: US 2022/0347325 A1
(43) Pub. Date: Nov. 3, 2022

(54) SANITIZING LAMP

(71) Applicant: OXTI CORPORATION, New Taipei City (TW)

(72) Inventor: Chih-Feng Ho, New Taipei City (TW)

(21) Appl. No.: 17/242,309

(22) Filed: Apr. 28, 2021

(51) Int. Cl. A61L 2/10

(52) U.S. Cl. CPC A61L 2/10; A61L 2202/11

(57) ABSTRACT

A sanitizing lamp includes: a lamp body, configured with a fixing part; a support, including a first connecting plate and second connecting plate coupled pivotally to each other, the first connecting plate configured with an accommodation groove, and the fixing part of the lamp body inserted and coupled pivotally inside the accommodation groove; and a base, including a standing frame and base frame coupled pivotally to each other, the second connecting plate of the support fixed to the standing frame. With the pivotal connection of the fixing part of the lamp body to the first connecting plate, with the pivotal connection of the first connecting plate to the second connecting plate, and with the pivotal connection of the standing frame to the base frame, thereby being convenient to adjust the illumination angle and illumination range of the lamp body.

### TECHNICAL FIELD OF THE INVENTION

The present invention relates to a sanitizing lamp, and more particularly to a sanitizing lamp capable of being adjusted in illumination angle and range.

### DESCRIPTION OF THE PRIOR ART

In the past, infectious diseases have changed the human history many times, and infectious diseases are one of the causes of most deaths in the world. With the change of human life style, the global climate change changes the transmission route and distribution area of infectious diseases; viruses and pathogenic microorganisms continue to adapt and mutate, causing every kind of emerging infectious diseases and re-emerging infectious diseases to happen incessantly; from early measles, smallpox, malaria, influenza to the recent severe acute respiratory syndrome (SARS), new type A influenza virus (H1N1), Middle East respiratory syndrome (MERS-CoV) and new coronavirus pneumonia (COVID-19), the currently known infectious transmission of such infectious diseases is mainly spread by direct contact with viral secretions or droplets.

Patients cause viruses to attach to doorknobs or door handles due to coughing, sneezing or contact with respiratory secretions, such that the doorknobs or door handles are highly contagious routes of the viruses. Taiwan Patent NO. M601622, titled “ultraviolet sanitizing lamp”, discloses a sanitizing lamp including a light head, base, flexible vertical pole, electric connector, and electric wire, where the light head includes a housing and a ultraviolet module installed on the housing; one end of the flexible vertical pole is in connection with the light head, and another end thereof the base. Since the illumination angle and illumination range of the sanitizing lamp are mainly achieved with the flexible vertical pole made of a bendable material such as rubber, silicone, thin plastics or thin metal, such that the adjustable illumination angle and range of the sanitizing lamp are very much limited.

### SUMMARY OF THE INVENTION

To overcome the above disadvantages, the present invention proposes a sanitizing lamp, including: a lamp body, configured with a fixing part; a support, including a first connecting plate and second connecting plate coupled pivotally to each other, the first connecting plate configured with an accommodation groove, and the fixing part of the lamp body inserted and coupled pivotally inside the accommodation groove; and a base, including a standing frame and base frame coupled pivotally to each other, the second connecting plate of the support fixed to the standing frame.

Preferably, the lamp body is configured with a signal receiver.

Preferably, the standing frame is configured with a magnetic element.

Preferably, the standing frame is provided with a screw fastener.

Preferably, the fixing part is provided with a power port.

With the pivotal connection of the fixing part of the lamp body to the first connecting plate, the lamp body is enabled to be pivoted relatively to the first connecting plate; with the pivotal connection of the first connecting plate to the second connecting plate, the first connecting plate is enabled to be pivoted relatively to the second connecting plate; and with the pivotal connection of the standing frame to the base frame, the standing frame is enabled to be pivoted relatively to the base frame, thereby being convenient to adjust the illumination angle and illumination range of the lamp body, and thus achieving the better illumination angle and illumination range, so as to improve the sterilization efficiency. In addition, the lamp body is further provided with the signal receiver to receive a wireless signal, which can remotely control the light emission.

### BRIEF DESCRIPTION OF THE DRAWINGS

FIG. 1 is an exploded view of a sanitizing lamp of the present invention;

FIG. 2 is a perspective view of the sanitizing lamp of the present invention;

FIG. 3 shows a power wire being passed through a wire arrangement slot;

FIG. 4 shows a magnetic element being configured with the wire arrangement slot;

FIG. 5 shows an illumination angle being adjusted to cause a lamp body to face downward;

FIG. 6 shows an illumination angle being adjusted to cause the lamp body to face upward;

FIG. 7 shows the pivoting of a support being adjusted and the lamp body being plugged and pulled to change an illumination direction;

FIG. 8 shows the illumination direction being adjusted to cause the lamp body to be turned to illuminate;

FIG. 9 shows the relative positions of a standing frame and base frame being adjusted to change an illumination range of the lamp body; and

FIG. 10 shows a watch being placed on the base frame for disinfection.

### DETAILED DESCRIPTION OF THE PREFERRED EMBODIMENTS

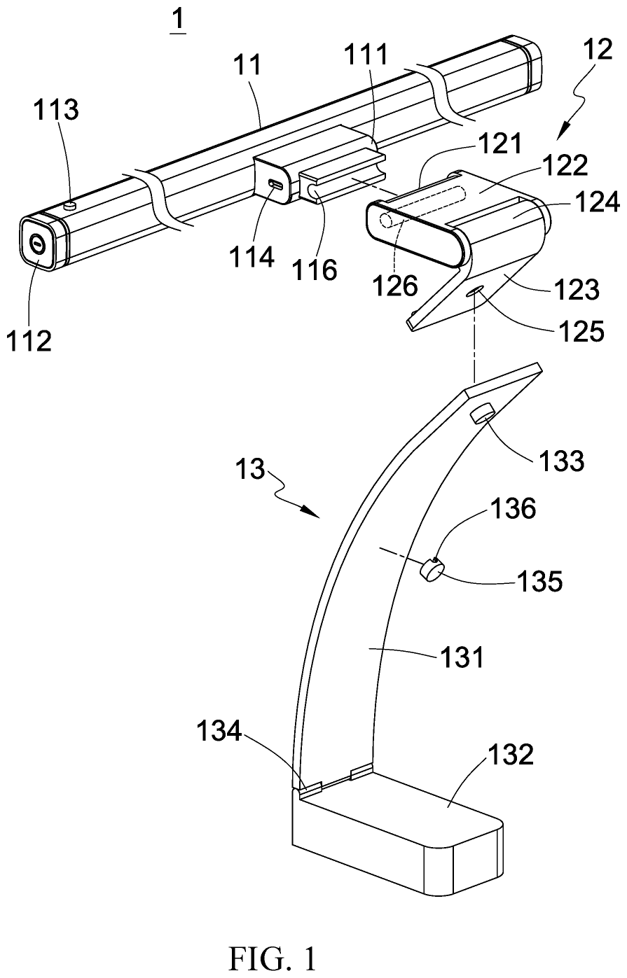

Referring to FIG. 1, a sanitizing lamp 1 of the present invention includes a lamp body 11, support 12 and base 13.

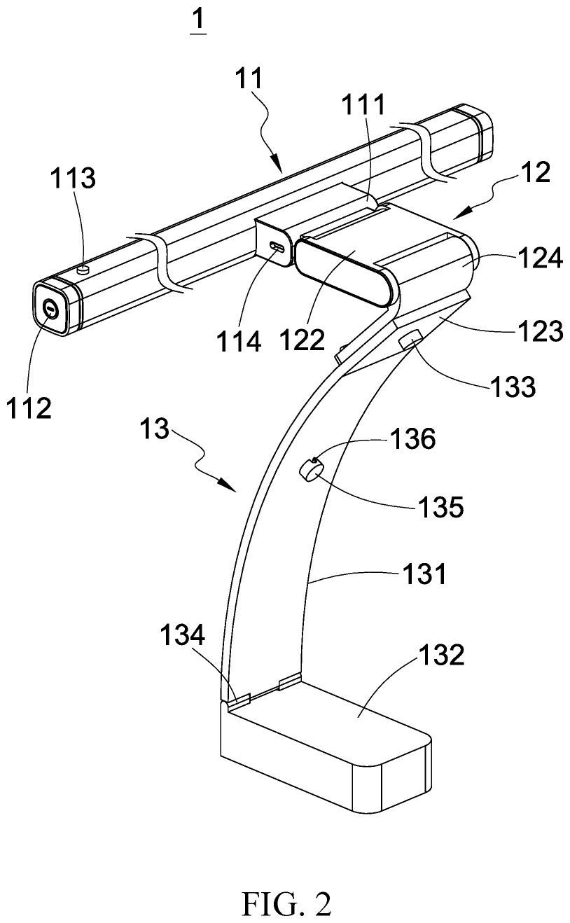

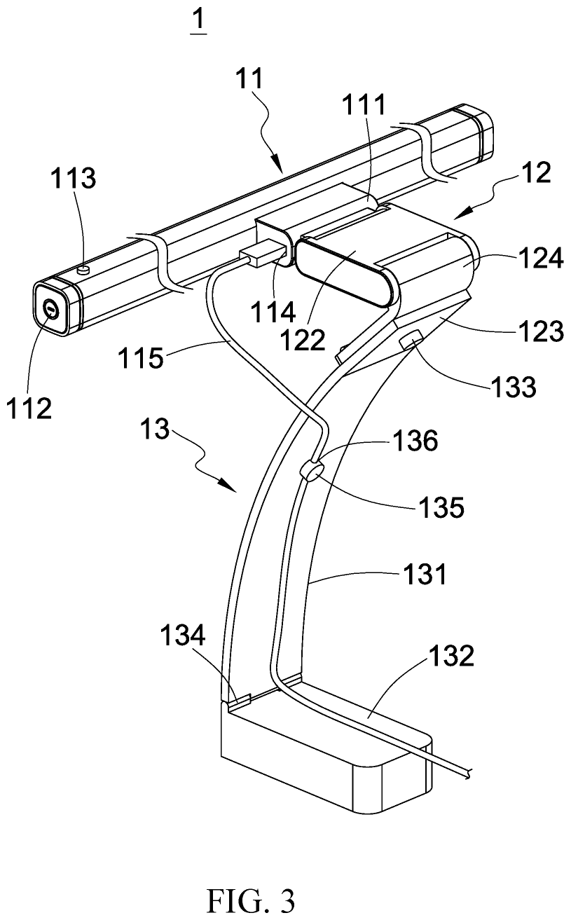

Referring to FIGS. 2 and 3, the lamp body 11 is configured with a fixing part 111, and may for example be a UVB lamp, UVC lamp, mercury lamp, metal halide lamp or xenon lamp or other electronic lamps with sterilization function. Furthermore, the lamp body 11 is configured with a switch key 112 to control lighting, and may be configured with a signal receiver 113 to receive a wireless signal to remotely control lighting. The fixing part 111 may be configured with a power port 114 to be in connection with a power wire 115 and thus an external power source. The fixing part 111 of the lamp body 11 may further be configured with a rotating shaft groove 116.

The support 12 includes a first connecting plate 122 and second connecting plate 123 coupled pivotally to each other, where between the first connecting plate 122 and second connecting plate 123 is configured a support rotating shaft 124, through which the first connecting plate 122 and second connecting plate 123 are coupled pivotally to each other, allowing the first connecting plate 122 to be pivoted relatively to the second connecting plate 123. The first connecting plate 122 is configured with an accommodation groove 121, inside which a lamp body rotating shaft 126 may be configured, and the fixing part 111 of the lamp body 11 is inserted in the accommodation groove 12, and coupled to the lamp body rotating shaft 126 through the rotating shaft groove 116, allowing the lamp body 11 to be assembled inside the accommodation groove 121 of the support 12 in a plug and pull way; the lamp body 11 and first connecting plate 122 are thus coupled pivotally to each other through the lamp body rotating shaft 126, allowing the lamp body 11 to be pivoted relatively to the first connecting plate 122.

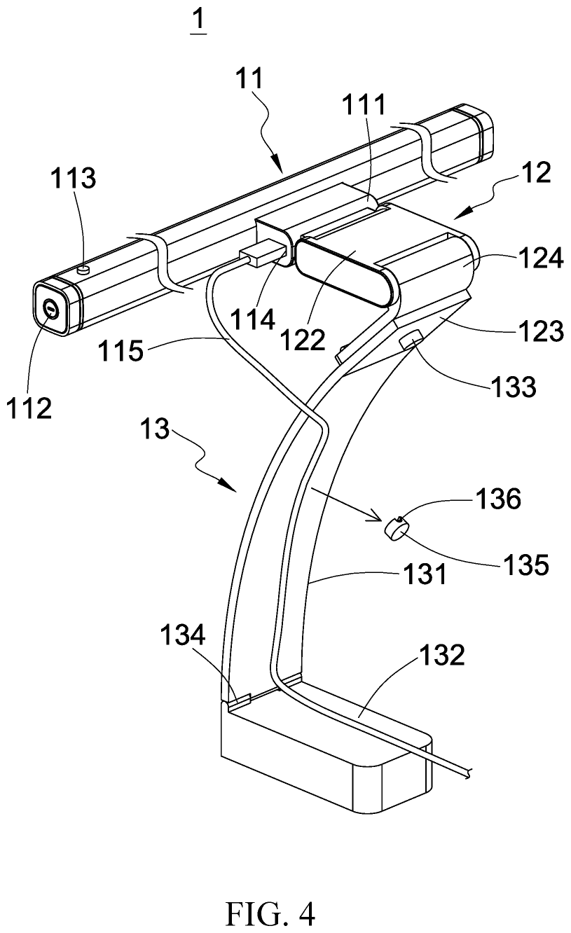

Referring to FIGS. 3 and 4, the base 13 includes a standing frame 131 and base frame 132 coupled pivotally to each other, and between the standing frame 131 and base frame 132 is configured a base rotating shaft 134, through which the standing frame 131 and base frame 132 are coupled pivotally to each other, allowing the standing frame 131 to be pivoted relatively to the base frame 132. The standing frame 131 may be configured with a screw fastener 133, which may for example a screw, corresponding to which the second connecting plate 123 of the support 12 is configured a screw hole 125, and the second connecting plate 123 of the support 12 can be fixed to the standing frame 131 by engaging the screw fastener 133 with the screw hole 125. In addition, the screw fastener 133 may be configured with a knurl, which allows the friction to be increased on the surface of the screw fastener 133 to facilitate holding. The standing frame 131 may further provided with a curved surface so as to achieve an eye-appealing effect through the appearance of streamlined lines. The standing frame 131 may further be provided with a magnetic element 135, and the magnetic element 135 may for example be a magnet and may be configured with a wire arrangement slot 136 adapted to allow the power wire 115 to be passed through, avoiding wire entanglement and thus achieving a certain function of line management, easy to organize.

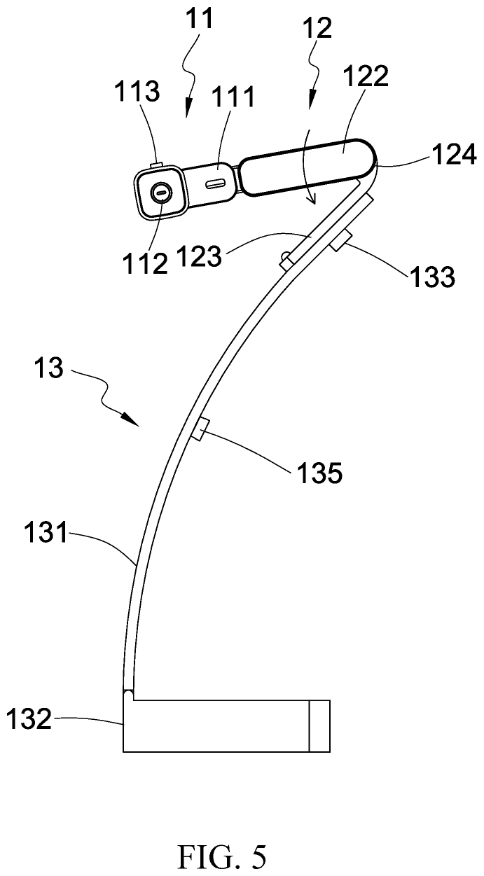

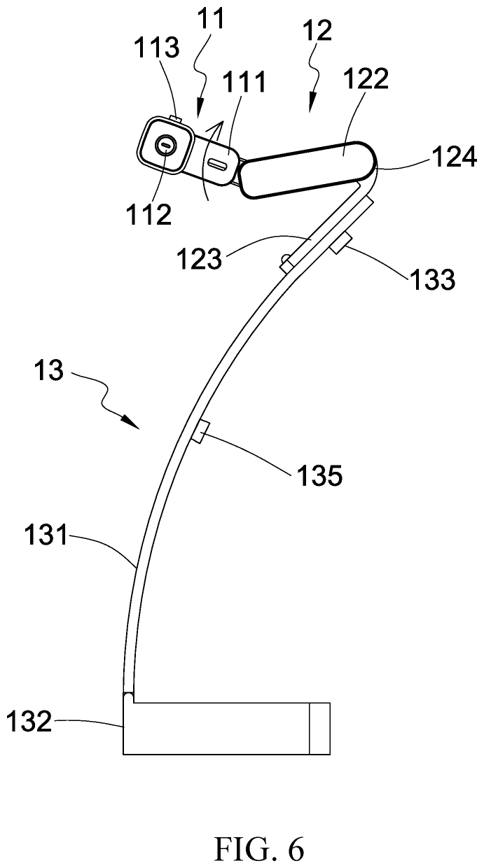

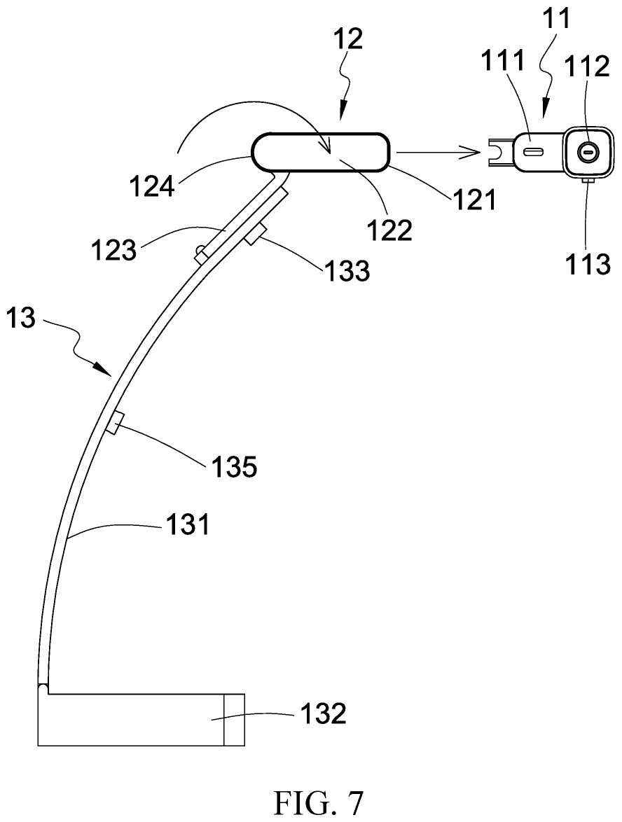

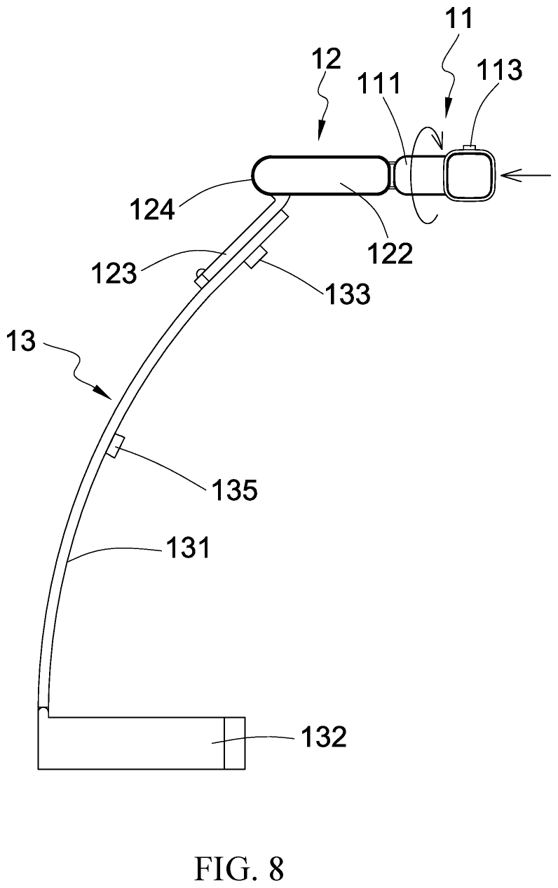

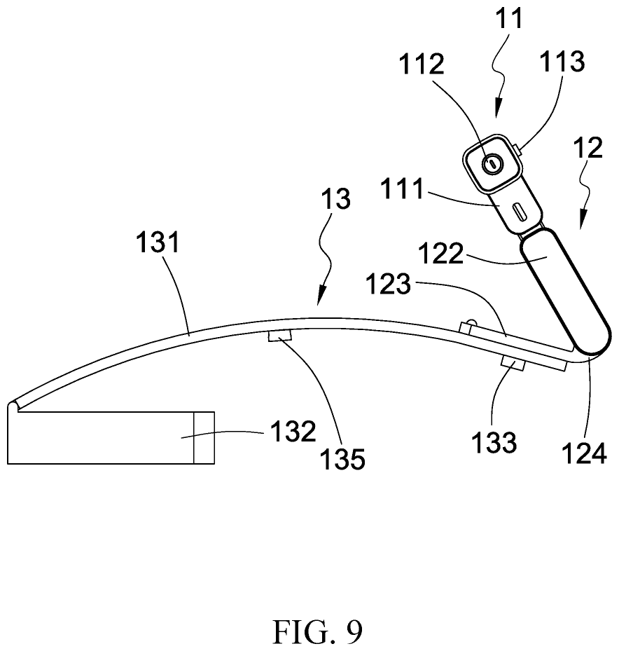

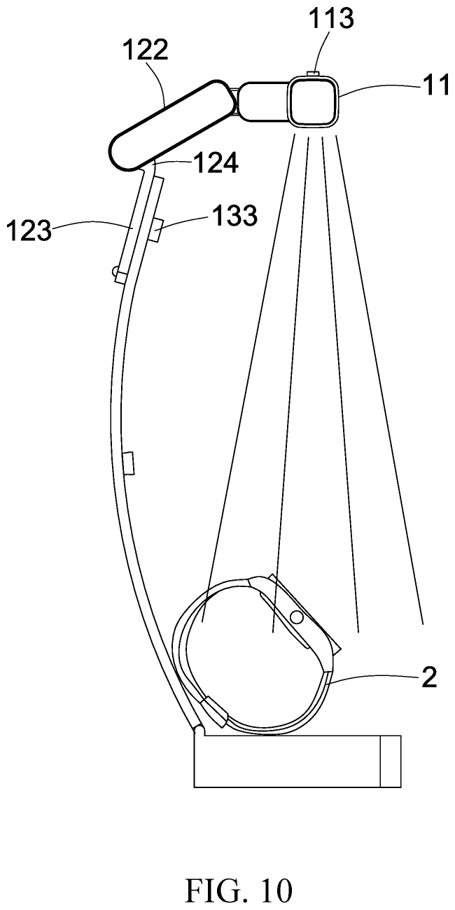

Referring to FIG. 5, the first connecting plate 122 is allowed to be pivoted relatively to the second connecting plate 123 through the pivotal coupling of the first connecting plate 122 to the second connecting plate 123. For example, the first connecting plate 122 is pivoted downward relatively to the second connecting plate 123, and the lamp body 11 is also pivoted downward so that the illumination angle and illumination direction (downward) of the lamp body 11 can thus be adjusted. Referring to FIG. 6, the lamp body 11 is pivoted relatively to the first connecting plate 122 through the pivotal connection of the lamp body 11 to the first connecting plate 122. For example, the lamp body 11 is pivoted upward relatively to the support 12, the illumination angle and illumination direction (upward) of the lamp body 11 can then be adjusted. Referring to FIGS. 7 and 8, the first connecting plate 122 is pivoted relatively to the second connecting plate 123 through the pivotal connection of the first connecting plate 122 to the second connecting plate 123, and the lamp body 11 is assembled inside the accommodation groove 121 of the support 12 in a plug and pull way. For example, the first connecting plate 122 is pivoted backward relatively to the second connecting plate 123, and the lamp body 11 is then pulled out from the accommodation groove 121 and plugged again in the accommodation groove 121 to change the illumination direction after being turned, and the illumination angle and illumination direction (backward) of the lamp body 11 can thus be adjusted. Referring to FIG. 9, the standing frame 131 is pivoted relatively to the base frame 132 through the pivotal connection of the standing frame 131 to the base frame 132, allowing the lamp body 11 to be in an inclined state to increase the illumination area of the lamp body 11, and further to change the illumination range of the lamp body 11. Referring to FIG. 10, an object such as a watch 2 can be placed on the base frame 132 for disinfection.

Conclusively, with the pivotal connection of the fixing part 111 of the lamp body 11 to the first connecting plate 122, the lamp body 11 is enabled to be pivoted relatively to the first connecting plate 122; with the pivotal connection of the first connecting plate 122 to the second connecting plate 123, the first connecting plate 122 is enabled to be pivoted relatively to the second connecting plate 123; and with the pivotal connection of the standing frame 131 to the base frame 132, the standing frame 131 is enabled to be pivoted relatively to the base frame 132, thereby being convenient to adjust the illumination angle and illumination range of the lamp body 11, and thus achieving the better illumination angle and illumination range, so as to improve the sterilization efficiency.

I claim:

1. A sanitizing lamp, comprising:
a lamp body, configured with a fixing part;
a support, comprising a first connecting plate and second connecting plate coupled pivotally to each other, said first connecting plate configured with an accommodation groove, and said fixing part of said lamp body inserted and coupled pivotally inside said accommodation groove; and
a base, comprising a standing frame and base frame coupled pivotally to each other, said second connecting plate of said support fixed to said standing frame.

2. The sanitizing lamp according to claim 1, wherein said lamp body is configured with a signal receiver.

3. The sanitizing lamp according to claim 1, wherein said standing frame is configured with a magnetic element.

4. The sanitizing lamp according claim 1, wherein said standing frame is provided with a screw fastener.

5. The sanitizing lamp according to claim 1, wherein said fixing part is provided with a power port.
